(19) United States
(12) Patent Application Publication
(10) Pub. No.: US 2004/0267030 A1
(43) Pub. Date: Dec. 30, 2004

(54) Novel process for the preparation of form 1 ranitidine hydrochloride

(75) Inventors: Wen J. Cheng, Midland, MI (US); Roger L. Briggs, Midland, MI (US); Lee V. McCoy, Midland, MI (US)

Correspondence Address: ROSS J. OEHLER, AVENTIS PHARMACEUTICALS INC., ROUTE 202-206, MAIL CODE: D303A, BRIDGEWATER, NJ, 08807, US

(73) Assignee: Aventis Pharmaceuticals Inc., Bridgewater, NJ

(21) Appl. No.: 10/894,169

(22) Filed: Jul. 19, 2004

Related U.S. Application Data
(63) Continuation of application No. 10/159,846, filed on May 31, 2002, now abandoned.
Application No. 10/159,846 is a continuation of application No. 09/668,203, filed on Sep. 22, 2000, now abandoned.
Application No. 09/668,203 is a continuation of application No. 09/360,871, filed on Jul. 23, 1999, now abandoned.
Application No. 09/360,871 is a continuation of application No. 09/070,446, filed on Apr. 30, 1998, now abandoned.
Application No. 09/070,446 is a continuation of application No. 08/822,168, filed on Mar. 21, 1997, now abandoned.
(60) Provisional application No. 60/042,579, filed on Feb. 26, 1997.

(51) Int. Cl. A61K 31/34; C07D 307/02

(52) U.S. Cl. 549/492

(57) ABSTRACT

The present invention provides a novel process for preparing Form 1 ranitidine hydrochloride by crystallization from at least one C₁-C₆ alkanol.

This application is a continuation of U.S. application Ser. No. 10/159,846, filed May 30, 2002, which is a continuation of Ser. No. 09/668,203, filed Sep. 22, 2000, which is a continuation of Ser. No. 09/360,871, filed Jul. 23, 1999, which is a continuation of Ser. No. 09/070,446, filed Apr. 30, 1998, now abandoned, which is a continuation of Ser. No. 08/822,168, filed Mar. 21, 1997, now abandoned, which claims the benefit of U.S. Provisional Application No. 60/042,579, filed Mar. 25, 2996.

The present invention relates to a novel process for preparing ranitidine hydrochloride, N-[2-[[[5-(dimethylamino)methyl-2-furanyl]methyl]thio]ethyl]-N′-methyl-2-nitro-1,1-ethenediamine hydrochloride, an antagonist of the H₂-histamine receptor which is useful in the treatment of gastric and peptic ulcers (U.S. Pat. No. 4,128,658, Dec. 5, 1978). More particularly, the present invention provides a novel process for preparing Form 1 ranitidine hydrochloride by crystallization from at least one C₁-C₆ alkanol.

Ranitidine hydrochloride is reported to exist in two polymorphic crystalline forms, designated Form 1 and Form 2. The preparation of Form 1 ranitidine hydrochloride is reported in U.S. Pat. No. 4,128,658. According to that preparation ranitidine hydrochloride is formed in a volume of industrial methylated spirits and the Form 1 polymorph is obtained upon precipitation or crystallization induced by the addition of an equal volume of ethyl acetate. However, Form 1 ranitidine hydrochloride prepared in this manner is reported to possess unsuitable filtration and drying characteristics, see U.S. Pat. No. 4,672,133.

The preparation of Form 1 ranitidine hydrochloride is also reported in U.S. Pat. No. 5,338,871. Accordingly, Form 1 ranitidine hydrochloride is prepared by crystallization, in the presence of seed crystals of pure Form 1 ranitidine hydrochloride, in a mixed solvent system comprising at least one C₁-C₄ alkanol and a C₆-C₁₀ aromatic hydrocarbon, the volume ratio of alkanol(s) to hydrocarbon being from about 1:1 to 1:2.

The preparation of Form 2 ranitidine hydrochloride is reported in U.S. Pat. No. 4,672,133. Accordingly, the Form 2 polymorph is prepared by crystallization from a solvent, in general, the solvent used is a lower alkanol. In a preferred preparation, a solution of ranitidine in propan-2-ol is treated with aqueous hydrochloric acid, followed by crystallization at elevated temperature at up to 70° C. by the addition of further quantities of isopropanol. Also, U.S. Pat. No. 4,672,133 teaches that the Form 2 polymorph is obtained by crystallization when the starting material is Form 1 or Form 2 ranitidine hydrochloride by dissolving the salt in a solvent, such as methanol or ethanol, by warming, followed by cooling of the resulting solution, e.g., to 10° to 40° C. and stirring.

We have now surprisingly discovered that Form 1 ranitidine hydrochloride is prepared by crystallization of ranitidine hydrochloride from at least one C₁-C₆ alkanol. Whereas the art teaches that Form 1 ranitidine hydrochloride is prepared in mixed solvent systems containing alkanols and antisolvents, such as ethyl acetate or a C₆-C₁₀ aromatic hydrocarbon, and that Form 2 ranitidine hydrochloride is formed upon crystallization from lower alkanols, it has been found according to the present invention that Form 1 ranitidine hydrochloride is obtained in pure polymorphic form by crystallizing ranitidine hydrochloride from at least one C₁-C₆ alkanol without a substantial amount of an antisolvent.

Thus, the present invention provides a process for preparing Form 1 ranitidine hydrochloride, comprising crystallization of ranitidine hydrochloride in at least one C₁-C₆ alkanol. The term “C₁-C₆ alkanol” refers to straight or branch chained alkanols containing from 1 to 6 carbon atoms, such as methanol, ethanol, n-propanol, isopropanol, n-butanol, t-butanol, i-butanol, s-butanol, n-pentanol, i-amyl alcohol, t-amyl alcohol, hexanol, and the like. According to the present invention preferred C₁-C₆ alkanols are ethanol, isopropanol, mixtures of ethanol and isopropanol, mixtures of methanol and isopropanol, and t-amyl alcohol, with ethanol and isopropanol being more preferred and isopropanol being most preferred.

Unlike the processes for preparing Form 1 ranitidine hydrochloride described in U.S. Pat. Nos. 4,128,658 and 5,338,871, the present process does not require the use of a substantial amount of an antisolvent. Thus, one aspect of the present invention is that it is generally carried out using at least one C₁-C₆ alkanol without an antisolvent. The term “antisolvent” refers to organic solvents in which ranitidine hydrochloride is not appreciably soluble and generally are of lower polarity than the alkanol(s) used to effect solution.

Polymorphs of ranitidine hydrochloride can be characterized by a variety of techniques, including differential scanning calorimetry, infra-red spectrometry, and x-ray diffraction, with varying sensitivity. H. G. Brittain, Pharmaceutical Technology, 50-52 (August 1994) and J. T. Carstensen and M. K. Franchini, Drug Development and Industrial Pharmacy, 21, 523-536 (1995). X-ray diffraction analysis provides a direct and sensitive method for distinguishing between Form 1 and Form 2 ranitidine hydrochloride.

Polymorphically pure Form 1 ranitidine hydrochloride is obtained by the present invention as determined by X-ray diffraction analysis using a Siemens D-500 automated powder diffractometer equipped with a Co x-ray tube source, primary beam monochromator, and a position sensitive detector, as follows: The incident beam is collimated using a 1° divergence slit. The active area on the position sensitive detector subtends approximately 5° 2θ. The source is operated at 35 kV and 30 mA and the samples are illuminated with Co Kα1 radiation, λ(Kα1)=1.788965 Angstroms. X-ray powder diffraction data are collected from 5° to 55° 2θ at a rate of 1° 2θ per minute with a step width of 0.02°2θ. The samples are rotated throughout data collection to maximize sampling statistics.

The present process can be carried out using ranitidine hydrochloride which is formed from a solution of ranitidine base in at least one C₁-C₆ alkanol by the addition of an appropriate amount of hydrogen chloride. An appropriate amount of hydrogen chloride is from about 0.8 to 1.2 molar equivalents, preferably 0.9 to 1.1 molar equivalents, more preferably 0.95 to 1.0 molar equivalents, and most preferably 1.0 molar equivalents. An appropriate amount of hydrogen chloride can be added as a gas or more conveniently as a solution in a C₁-C₆ alkanol. Alternately, the present process can be carried out using preformed ranitidine hydrochloride.

It has been found that in the present process the rate of nucleation from the initial solution is important in obtaining polymorphically pure Form 1 ranitidine hydrochloride. The formation of Form 1 ranitidine hydrochloride being favored by more rapid nucleation. The rate of nucleation, and thus polymorphic composition and purity of the crystals formed, is affected by the temperature of the initial solution, by the water content of the initial solution, by the concentration of ranitidine hydrochloride in the initial solution, and by seeding.

According to the present process, Form 1 ranitidine hydrochloride may be obtained when the temperature of the initial solution is as low as 0° C. In addition, it is well known that temperatures above 70° C. are not well tolerated by ranitidine hydrochloride in solution. Thus, at least in the initial stages, i.e., before nucleation, the present process is carried out at temperatures of from about 0° C. to about 70° C., with temperatures of from about 0° C. to about 60° C. being preferred and temperatures of from about 10° C. to about 40° C. being more preferred.

Also, Form 1 ranitidine hydrochloride may be obtained when the water content of the initial solution before nucleation is less than about 0.7%. Thus, for the present process the water content of the initial crystallization solution is less than about 0.7%, preferably less than about 0.4% and more preferably less than about 0.3%.

The rate of nucleation is most greatly affected by the concentration of ranitidine hydrochloride in the initial solution. Thus, the concentration of ranitidine hydrochloride in solution before nucleation is more important than temperature and water content in determining the polymorphic purity of Form 1 ranitidine hydrochloride obtained by the present invention, i.e., the formation of Form 1 ranitidine hydrochloride from less concentrated solutions is more sensitive to water content and temperature than it is from more concentrated solutions.

Seeding the initial solution with Form 1 ranitidine hydrochloride crystals is effective in insuring rapid nucleation to give Form 1 ranitidine hydrochloride. Of course, it is understood to be most effective, the crystallization solution should be seeded with pure Form 1 ranitidine hydrochloride before nucleation. Seeding the crystallization solution with Form 1 ranitidine hydrochloride crystals is a preferred embodiment of the present invention.

After nucleation of Form 1 ranitidine hydrochloride from the initial solution, Form 1 ranitidine hydrochloride continues to crystallize. Unlike the initial solution, it has been found that after nucleation, Form 1 ranitidine hydrochloride continues to form even though water is added, the temperature is lowered below 0° C., or both water is added and the temperature is lowered below 0° C.

The filtering and drying characteristics of the Form 1 ranitidine hydrochloride obtained from the present process are affected by the size of the crystals formed. Smaller crystals have less desirable filtering and drying characteristics. It has been found that the size of the Form 1 ranitidine hydrochloride crystals obtained by the present invention can be increased by digestion techniques giving Form 1 ranitidine hydrochloride possessing good filtration and drying characteristics.

Upon nucleation the crystals of Form 1 ranitidine hydrochloride formed may be very small. Digestion of the crystals formed upon nucleation allows larger crystals to grow at the expense of smaller crystals. J. W. Mullin, Crystallization (1961). Digestion can be achieved, as is known in the art, by reducing the population of crystals in the crystallization solution, either by elevating the temperature of the solution or by the addition of a digestion solvent. In a preferred embodiment of the present invention the crystals formed upon nucleation are digested. It is preferred that the digestion is carried out by the addition of a digestion solvent, such as water, methanol, or ethanol, with water being preferred.

The foregoing process is exemplified by the procedures given below. These procedures are understood to be illustrative only and are not intended to limit the scope of the invention in any way. As used in these procedures, the following terms have the meanings indicated: “g” refers to grams; “kg” refers to kilograms; “mmol” refers to millimoles; “mol” refers to moles; “mL” refers to milliliters; “L” refers to liters; “° C.” refers to degrees Celsius.

### EXAMPLE 1

Preparation of Form 1 ranitidine hydrochloride

Combine ranitidine base (1.0 g, 3.2 mmol) and isopropanol (6.2 g). Add a solution of hydrogen chloride in isopropanol (3.2 g, 4% by weight, 3.5 mmol) to form an oil. Warm to about 53° C. Slowly cool to about 26° C. over about 1.5 hours to give a solid. Collect the solid by filtration, and dry in vacuo to give the title compound.

### EXAMPLE 2

Preparation of Form 1 ranitidine hydrochloride

Combine ranitidine base (24.6 g, 78.3 mmol) and isopropanol (120.4 g). Warm to 33° C. Add a solution of hydrogen chloride in isopropanol (28.5 g, 10% by weight, 78.0 mmol) over about 9 minutes to give an emulsion. Stir the emulsion to give a solid. Warm to about 48° C. Cool to about 20° C. Collect the solid by filtration, and dry in vacuo to give the title compound.

### EXAMPLE 3

Preparation of Form 1 ranitidine hydrochloride

Combine ranitidine base (66 g, 210 mmol) and t-amyl alcohol (251 g). Warm to 51° C. Slowly, add a solution of hydrogen chloride in isopropanol (20.5 g, 15% by weight, 84 mmol) and stir to give a solid. After 18 hours, collect the solid by filtration and dry in vacuo to give the title compound.

### EXAMPLE 4

Preparation of Form 1 ranitidine hydrochloride

Combine ranitidine base (30.0 g, 95.4 mmol), isopropanol (256.5 g), and methanol (13.5 g) at 30° C. Add seed crystals of Form 1 ranitidine hydrochloride (0.065 g). With stirring, add a solution of hydrogen chloride in isopropanol (10% by weight) at a rate of about 0.97 g per minute until about 17.5 g of the solution is added. After about 4 hours, continue adding the solution of hydrogen chloride in isopropanol at a rate of 0.05 g per minute until 34.8 g (total) of the solution is added. Collect the solid by filtration, rinse with cold isopropanol, and dry in vacuo to give the title compound.

### EXAMPLE 5

Preparation of Form 1 ranitidine hydrochloride

Combine solid ranitidine hydrochloride (1.01 g, 3.2 mmol) and ethanol (12.6 g) containing about 0.27% water. Warm to dissolve. Seed with Form 1 ranitidine hydrochloride at about 36° C. After 4.5 hours at 36° C., collect the solid by filtration and dry in vacuo to give the title compound. Karl Fisher determination of the mother liquor gives 0.46% water.

### EXAMPLE 6

Preparation of Form 1 ranitidine hydrochloride

Combine ranitidine base (2.0 g, 6.36 mmol), isopropanol (14.05 g) and warm to about 30° C. With stirring, add a solution of hydrogen chloride in isopropanol (5% by weight) at a rate of about 0.15 g per minute until about one fourth the solution is added. Add seed crystals of Form 1 ranitidine hydrochloride. Continue adding the solution of hydrogen chloride in isopropanol at a rate of 0.15 g per minute until 4.95 g (total) of the solution is added. Collect the solid by filtration and dry in vacuo to give the title compound. Karl Fisher determination of the mother liquor gives 0.31% water.

### EXAMPLE 7

Preparation of Form 1 ranitidine hydrochloride

Combine ranitidine base (1.0 g, 3.18 mmol), isopropanol (7.40 g) and warm to about 50° C. With stirring, add a solution of hydrogen chloride in isopropanol (5% by weight) at a rate of about 0.15 g per minute until about one fourth the solution is added. Add seed crystals of Form 1 ranitidine hydrochloride. Continue adding the solution of hydrogen chloride in isopropanol at a rate of 0.15 g per minute until 2.50 g (total) of the solution is added. Collect the solid by filtration and dry in vacuo to give the title compound.

### EXAMPLE 8

Preparation of Form 1 ranitidine hydrochloride

Combine ranitidine base (1.0 g, 3.18 mmol), isopropanol (7.40 g) and warm to about 60° C. With stirring, add a solution of hydrogen chloride in isopropanol (5% by weight) at a rate of about 0.15 g per minute until about one fourth the solution is added. Add seed crystals of Form 1 ranitidine hydrochloride. Continue adding the solution of hydrogen chloride in isopropanol at a rate of 0.15 g per minute until 2.50 g (total) of the solution is added. Cool to about 25° C., collect the solid by filtration, and dry in vacuo to give the title compound.

### EXAMPLE 9

Preparation of Form 1 ranitidine hydrochloride

Combine ranitidine base (20.0 g, 63.6 mmol) and isopropanol (146.3 g). Warm to 40° C. With stirring, add a solution of hydrogen chloride in isopropanol (4.9% by weight) at a rate of 0.74 g per minute. After about 8 g of solution of hydrogen chloride in isopropanol is added, add seed crystals of Form 1 ranitidine hydrochloride. Continue adding the solution of hydrogen chloride in isopropanol until about 14 g of the solution is added. Stir until nucleation is complete and then add water (11.8 g). After about 1 to 2 hours, resume the addition of the solution of hydrogen chloride in isopropanol by increasing the flow rate gradually from about 0.036 g per minute to about 0.25 g per minute until 47.6 g, 64 mmol (total) of the solution has been added. Cool the slurry obtained to about 13° C., collect the solid by filtration, and dry in vacuo to give the title compound.

### EXAMPLE 10

Preparation of Form 1 ranitidine hydrochloride

Combine ranitidine base (800 g, 2.54 mol) and isopropanol (5.86 kg). Warm to 44° C. With stirring, add a solution of hydrogen chloride in isopropanol (5% by weight) at a rate of 8 g per minute until about 150 g of the solution is added. Add seed crystals of Form 1 ranitidine hydrochloride (0.095 g). Continue adding the solution of hydrogen chloride in isopropanol at a rate of 8 g per minute until 406 g (total) of the solution is added. After nucleation is complete (about 4 hours), add water (210.5 g) to the slurry. After about 40 minutes, resume the addition of the solution of hydrogen chloride in isopropanol at 1.0 g per minute until an additional 1387 g (1793 g, total) of the solution is added. Cool the slurry obtained to about 10° C., collect the solid by filtration, rinse with cold isopropanol, and dry in vacuo to give the title compound.

### EXAMPLE 11

Preparation of Form 1 ranitidine hydrochloride

Combine ranitidine base (2.9 kg, 9.22 mol) and isopropanol (8.25 kg) at 37° C. Filter thorough a 1 micron filter and rinse the filter with isopropanol (0.45 kg). Add seed crystals of Form 1 ranitidine hydrochloride (0.31 g in 0.97 g of isopropanol). Warm to 55° C. With stirring, add a solution of hydrogen chloride in isopropanol (15% by weight) at a rate of about 19 g per minute until about 0.9 kg of the solution is added. Hold the temperature of the crystallization solution at about 55° C. After nucleation is complete (about 6 hours), add water (0.53 kg). After digestion is complete (about 1 hour), continue adding the solution of hydrogen chloride in isopropanol at a rate of 4 g per minute until 2.24 kg (total) of the solution is added. Cool the slurry obtained to about 10° C., collect the solid by filtration, rinse with cold isopropanol, and dry in vacuo to give the title compound.

### EXAMPLE 12

Preparation of Form 1 ranitidine hydrochloride

Combine ranitidine base (30.2 g, 96 mmol) and isopropanol (210.5 g). Filter thorough a 1 micron filter and rinse the filter with isopropanol (60 g). Cool to about 14° C. Add seed crystals of Form 1 ranitidine hydrochloride (0.044 g). With stirring, add a solution of hydrogen chloride in isopropanol (10% by weight) at a rate of 1.33 g per minute until about 17.4 g of the solution is added. After nucleation is complete (about 4.5 hours), warm to about 35° C. and add water (15 g). After about 1 hour, continue adding the solution of hydrogen chloride in isopropanol at a rate of 0.033 g per minute until 34.8 g (total) of the solution is added. Cool the slurry obtained to about 11° C., collect the solid by filtration, rinse with cold isopropanol, and dry in vacuo to give the title compound.

### EXAMPLE 13

Preparation of Form 1 ranitidine hydrochloride

Combine ranitidine base (100 g, 318 mmol) and isopropanol (358.6 g). Cool to about 15° C. Add seed crystals of Form 1 ranitidine hydrochloride. With stirring, add a solution of hydrogen chloride in isopropanol (15% by weight) at a rate of 1.13 g per minute until about 38.6 g of the solution is added. After nucleation is complete (about 2 hours), warm to about 35° C. and add water (22.2 g). After about 30 minutes, continue adding the solution of hydrogen chloride in isopropanol at a rate of 0.079 g per minute until an additional 38.6 g of the solution is added. Cool the slurry obtained to about 5° C., collect the solid by filtration, and dry in vacuo to give the title compound.

What is claimed is:

1. A method for processing Form 1 ranitidine hydrochloride crystals from a post nucleation solution of ranitidine hydrochloride wherein the improvement comprises preparing the post nucleation solution, from a pre-nucleation solution containing ranitidine hydrochloride and consisting essentially of a solvent of at least one C₁-C₆ alkanol having a water content of less than about 0.7% and having a temperature from about 0° C. to about 70° C., by allowing the nucleation to commence therefrom, and then adding a digestion solvent to the post nucleation solution.

2. The method according to claim 1 wherein the C₁-C₆ alkanol is isopropanol.

3. The method according to claim 1 wherein the C₁-C₆ alkanol is ethanol.

4. The method according to claim 1 wherein the solution of ranitidine hydrochloride in the solvent is prepared by dissolving ranitidine base in the solvent containing from about 0.8 to about 1.2 molar equivalents of hydrogen chloride and wherein the method is carried out at temperatures from about 0° C. to about 70° C.

5. The method according to claim 4 wherein the C₁-C₆ alkanol is isopropanol.

6. The method according to claim 5 wherein the digestion solvent is water.

7. The method of claim 4 further comprising collecting Form 1 ranitidine hydrochloride and wherein the method is carried out at temperatures from about 0° C. to about 60° C.

8. The method according to claim 7, wherein the ranitidine base is dissolved in the C₁-C₆ alkanol solvent at a temperature of about 60° C.

9. The method according to claim 1 wherein the solution of ranitidine hydrochloride in the solvent is prepared by dissolving ranitidine hydrochloride in the C₁-C₆ alkanol solvent and wherein the method is carried out at temperatures from about 0° C. to about 70° C.

10. The method according to claim 9, wherein the ranitidine hydrochloride dissolved in the C₁-C₆ alkanol solvent is Form 2 ranitidine hydrochloride.

11. The method according to claim 9, wherein the ranitidine hydrochloride is dissolved in the C₁-C₆ alkanol solvent at a temperature of from about 0° C. to about 40° C.

12. The method according to claim 4 wherein the solution of ranitidine hydrochloride in the solvent is prepared by dissolving ranitidine base in isopropanol containing 1.08 molar equivalents of hydrogen chloride and where the method is carried out at a temperature of about 60° C.

13. The method according to claim 4 wherein the solution of ranitidine hydrochloride in the solvent is prepared by dissolving ranitidine base in isopropanol containing 1.00 molar equivalents of hydrogen chloride and where the method is carried out at a temperature of about 40° C.

14. The method according to claim 4 wherein the solution of ranitidine hydrochloride in the solvent is prepared by dissolving ranitidine base in isopopanol containing 1.00 molar equivalents of hydrogen chloride and wherein the method is carried out a temperature of about 14° C.

15. The method according to claim 9 wherein the solution of ranitidine hydrochloride in the solvent is prepared by dissolving ranitidine hydrochloride in ethanol having a water content of less than 0.27% and where the method is carried out and maintained at a temperature of about 36° C.

16. The method according to claim 1 wherein the nucleation of Form 1 ranitidine hydrochloride crystals is initiated using Form 1 ranitidine hydrochloride seed crystals.
